(19) United States
(12) Patent Application Publication
(10) Pub. No.: US 2022/0267795 A1
(43) Pub. Date: Aug. 25, 2022

(54) COMPOSITIONS AND METHODS FOR REGULATING PRODUCTION OF AN ANGIOGENSIS INHIBITOR

(71) Applicant: Kinase Pharma Inc., Calgary (CA)

(72) Inventor: Bradley G. THOMPSON, Calgary (CA)

(21) Appl. No.: 17/183,936

(22) Filed: Feb. 24, 2021

(51) Int. Cl. C12N 15/86; C07K 16/22; C12N 15/67

(52) U.S. Cl. CPC C12N 15/86; C07K 16/22; C12N 15/67; C12N 2750/14143

(57) ABSTRACT

The present disclosure relates to one or more agents, therapies, treatments, and methods of use of the agents and/or therapies and/or treatments for increasing production of a Bevacizumab like protein (BLP) by a subject that is administered the agent, therapy or treatment. Embodiments of the present disclosure can be used as a therapy or a treatment for a subject that has a condition that may benefit from reducing the formation of new blood vessels in one or more populations of high metabolic-rate cells, such as tumor cells.

### TECHNICAL FIELD

The present disclosure generally relates to compositions and methods for regulating angiogensis. In particular, the present disclosure relates to compositions and methods for regulating endogenous production of an inhibitor of growth factors that facilitate angiogensis.

### BACKGROUND

Cells with high metabolic rates can become hypoxic when the use of oxygen and other metabolic input compounds exceed the supply. Examples of such cells with high metabolic rates include tumor cells. Hypoxia can trigger increased production of vascular endothelial growth factor (VEGF) protein, which is released into the systemic circulatory system to act both locally and distally. VEGE can bind to specific membrane hound receptors of endothelial cells and this binding event may result in increased growth and recruitment of endothelial cells and, ultimately, in new blood vessels being developed. Greater blood vessel density results in increased blood flow and, ultimately, decreased hypoxia experienced by the high metabolic rate cells.

A known method for decreasing the growth and/or proliferation of high metabolic rate cells, such as tumor cells, is to interfere with the activity of VEGF. For example, a recombinant human monoclonal antibody, referred to as Bevacizumab, can be administered to a subject. This antibody can enter the subject's circulatory system where it binds to VEGF proteins and inhibits VEGF activity. The inhibiting of VEGF activity decreases VEGF-mediated angiogenesis, which the high metabolic-rate cells require to continue growing. This interrupts—at least temporarily—tumor cell and tissue growth. Bevacizumab is also known to enhance the efficacy of other chemotherapy compound(s). For example, when used in combination, Bevacizumab can, inter alia, enhance endothelial cell permeability and, in turn, enhance access of the other chemotherapy compound(s) into the tumor cell targets.

On addition to its known clinical side effects, Bevacizumab treatment has known drawbacks including, but not limited to, that it is an expensive per-dose treatment, that its benefits are reversible over time and, therefore, that multiple doses are required.

### SUMMARY

Some embodiments of the present disclosure relate to compositions and methods that cause a subject to produce a Bevacizumab-like protein (BLP). In some embodiments of the present disclosure, the subject's production of the BLP is endogenous. The BLP may be bioavailable and functionally equivalent to an exogenously delivered Bevacizumab.

In some embodiments of the present disclosure, the compositions described herein comprise a vector of plasmid deoxyribonucleic acid (DNA) that includes an insert sequence of nucleic acids. The insert sequence encodes for the production of the BLP and the insert sequence may also include a backbone sequence of nucleic acids that facilitates introduction of the insert sequence into one or more of a subject's cells. Within the subject's cells, the insert sequence is expressed and/or replicated. Expression of the insert sequence by one or more cells of the subject results in an increased production of the BLP by the subject. In some embodiments of the present disclosure, the methods that upregulate the production of BLP and to methods of manufacturing and administering the compositions that result in a subject's increased production of BLP.

Sonic embodiments of the present disclosure relate to compositions and methods that can be used as a therapy or a treatment for a subject that has a condition associated with increased growth and/or proliferation of high metabolic rate cells, such as tumor cells. The embodiments of the present disclosure may result in a subject Who receives such therapy or treatment to increase production of the BLP. The BLP may interfere with the activity of vascular endothelial growth factor (VEGF). Some embodiments of the present disclosure relate to a recombinant virus vector (RVV) that forms part of such a therapy or treatment. The RVV comprises a nucleotide sequence encoding production of the BLP.

Some embodiments of the present disclosure relate to a composition that comprises a nucleotide sequence according to the present disclosure (SEQ ID No. 9) of which at least a portion can be expressed in a target cell.

Some embodiments of the present disclosure relate to an insert for use with an RVV, wherein the insert has a nucleotide sequence has one or more of SEQ ID: No. 1, SEQ. ID No. 2, SEQ ID No. 3, SEQ ID No. 4, SEQ ID No. 5, SEQ ID No. 6, SEQ ID No. 7, and/or SEQ ID No. 8.

Some embodiments of the present disclosure relate to the insert with one or more of SEQ. ID No. 4, SEQ ID No. 5, SEQ ID No. 6, SEQ ID No. 7, and/or SEQ ID No. 8 for use with an RVV.

Some embodiments of the present disclosure relate to a method of making an agent/target cell complex, the .method comprising a step of administering a therapeutically effective amount of the agent to a subject, wherein the agent/target cell complex increases the subject's production of the BLP.

Some embodiments of the present disclosure relate to a pharmaceutical agent that comprises an agent, a pharmaceutically acceptable carrier and/or an excipient. Administering the pharmaceutical agent to a subject may increase the subject's production of the BLP.

Some embodiments of the present disclosure relate to a method of treating a condition. The method comprises a step of administering to a subject a therapeutically effective amount of an agent that upregulates the subject's production of the BLP and the BLP may ameliorate the condition. In some embodiments of the present disclosure, the condition is cancer.

Some embodiments of the present disclosure relate to a use of an agent for treating a condition, wherein the agent upregulates the subject's production of the BLP and the. BLP may ameliorate the condition. In some embodiments of the present disclosure, the condition is cancer.

Embodiments of the present disclosure relate to at least one approach for inducing endogenous production of the BLP. A first approach utilizes one or more gene vectors containing nucleotide sequences for increasing the endogenous production of the BLP. The one or more vectors can be administered to a subject to increase the subject's production of the BLP.

In some embodiments of the present disclosure, following administration of the agent the BLP may be produced within the subject's cells as a precursor BLP protein that can be subjected to one or more post-translational modification processes, which results in subject cells that are producing the precursor BLP protein to produce a final BLP product that is bioavailable and functional. In some embodiments of the present disclosure, the BLP product may be capable of participating in a binding event with a ligand for a specific family of receptor proteins. Without being bound by any particular theory, when such a binding event occurs, the BLP product can act to prevent the ligand from binding to and for activating the family of receptor proteins. For example, the ligand may be vascular endothelial growth factor (VEGF) and the BLP can bind thereto. When the BLP and VEGF ligand are bound together, that may reduce the recruitment and growth of endothelial cells and, ultimately, a reduced formation of new blood vessels within high metabolic rate cells. At least one non-limiting example of high metabolic rate cells is tumor cells.

### BRIEF DESCRIPTION OF THE DRAWINGS

These and other features of the present disclosure will become more apparent in the following detailed description in which reference is made to the appended drawings.

FIG. 1 is a scatter plot that shows human IgG expression in mice following administration of a vector, according to embodiments of the present disclosure, in mice with tumor cells.

FIG. 2 is a Kaplan-Meier plot for mice that were administered a control or a treatment, according to embodiments of the present disclosure.

### DETAILED DESCRIPTION

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “activity” is used interchangeably with the term “functionality” and both terms refer to the physiologic action of biomolecule.

As used herein, the term “agent” refers to a substance that, when administered to a subject, causes one or more chemical reactions and/or one or more physical reactions and/r or one or more physiological reactions and/or one or more immunological reactions in the subject. In some embodiments of the present: disclosure, the agent is a plasmid vector.

As used herein, the term “ameliorate” refers to improve and/or to make better and/or make more satisfactory.

As used herein, the term “biomolecule” refers to a carbohydrate, a protein, an amino acid sequence, a nucleic acid, a lipid, a primary metabolite, a secondary metabolite or another metabolite that is found within a subject. A biomolecule may be endogenous or exogenous to a subject.

As used herein, the term “cell” refers to a single cell as well as a plurality of cells or a population of the same cell type or different cell types. Administering an agent to a cell includes in vivo, in vitro and ex vivo administrations and/or combinations thereof.

As used herein, the term “complex” refers to an association, either direct or indirect, between one or more particles of an agent and one or more target cells. This association results in a change in the metabolism of the target cell. As used herein, the phrase “change in metabolism” refers to an increase or a decrease in the one or more target cells' production of deoxyribonucleic acid (DNA), ribonucleic acid (RNA), one or more proteins, an/or any post-translational modifications of one or more proteins.

As used herein, the terms “dysregulation” and “dysregulated” refer to situations or conditions wherein homeostatic control systems have been disturbed and/or compromised so that one or more metabolic, physiologic and/or biochemical systems. within a subject operate partially or entirely without said homeostatic control systems.

As used herein, the term “effector molecule” refers to a molecule within a subject that can directly or indirectly regulate the metabolic activity of a target cell by increasing or decreasing the production of DNA, RNA and or amino-acid sequences and/or by increasing or decreasing any post-translational modifications of one or more proteins.

As used herein, the term “endogenous” refers to the production and/or modification of a molecule that originates within a cell of a subject.

As used herein, the term “excipient” refers to any substance, not itself an agent, which may be used as a component within a pharmaceutical composition or a medicament for administration of a therapeutically effective amount of the agent to a subject. Additionally, or alternatively, an excipient may, either alone or in combination with further chemical components, improve the handling and/or storage properties and, permit or facilitate formation of a dose unit of the agent. Excipients include, but are not limited to, one or more of a binder, a disintegrant, a diluent, a buffer, a taste enhancer, a solvent, a thickening agent, a gelling agent, a penetration enhancer, a solubilizing agent, a wetting agent, an antioxidant, a preservative, a surface active agent, a lubricant, an emollient, a substance that is added to mask or counteract a disagreeable odor, fragrance or taste, a substance added to improve appearance or texture of the composition and/or substance that is used to form the pharmaceutical compositions or medicaments. Any such excipients can be used in any dosage forms according to the present disclosure. The foregoing classes of excipients are not meant to be exhaustive but are provided merely to be illustrative of what a person of skill in the art would know and would also recognize that additional types and combinations of excipients may be used to achieve delivery of a therapeutically effective amount of the agent to a subject through one or more routes of administration.

As used herein, the term “exogenous” refers to a molecule that is within a subject but that did not originate within the subject.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a. 10%, 20%, 30%, 40%, 50%, 60%, 70%, 80%, 90%, 100%, or any amount of reduction in between the specifically recited percentages, as compared to native or control levels.

As used herein, the term “medicament” refers to a medicine and car pharmaceutical composition that comprises the agent and that can promote recovery from a disease, disorder or symptom thereof and, or that can prevent a disease, disorder or symptom thereof and or that can inhibit the progression of a disease, disorder, or symptom thereof.

As used herein, the term “patient” refers to a subject that is afflicted with a disease or disorder. The term “patient” includes human and veterinary subjects.

As used herein, the term “pharmaceutical composition” means any composition comprising, bat not necessarily limited to, an agent to be administered a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anaesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the agent. Some examples of pharmaceutically acceptable carriers and their formulations are described in Remington (1995, The Science and Practice of Pharmacy (19th ed.) ed. A. R. Gennaro, Mack Publishing Company, Easton, Pa.), the disclosure of which is incorporated herein by reference. Typically, an appropriate amount of a pharmaceutically acceptable carrier is used in the formulation to render said formulation isotonic. Examples of suitable pharmaceutically acceptable carriers include, hut are not limited to: saline solutions, glycerol solutions, ethanol, N-(1(2,3-dioleyloxy)propyl)-N,N,N-trimethylammonium chloride (DOTMA), dioleolphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation should suit the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevention of” and “preventing” refer to avoiding the onset or progression of a disease, disorder, or a symptom thereof.

As used herein, the terms “production”, “producing” and “produce” refer to the synthesis and/or replication of DNA, the transcription of one or more sequences of RNA, the translation of one or more amino acid sequences, the post-translational modifications of an amino-acid sequence, and/or the production of one or more regulatory molecules that can influence the production and/or functionality of an effector molecule or an effector cell. For clarity, “production” is also be used herein to refer to the functionality of a regulatory molecule, unless the context reasonably indicates otherwise.

As used herein, the terms “promote”, “promotion”, and “promoting” refer to an increase in an activity, response, condition, disease process, or other biological parameter. This can include, but is not limited to, the initiation of the activity, response, condition, or disease process. This may also include, for example, a 10% increase in the activity, response, condition, or disease as compared to the native or control level. Thus, the increase in an activity, response, condition, disease, or other biological parameter can be 10%, 20%, 30%, 40%, 50%, 60%, 70%, 80%, 90%, 100%, or more, including any amount of increase in between the specifically recited percentages, as compared to native or control levels.

As used herein, the term “prophylactic administration” refers to the administration of any composition to a subject, in the absence of any symptom or indication of a disease or disorder, to prevent the occurrence and/or progression of the disease or disorder within the subject.

As used herein, the terms “signal molecule”, “signalling molecule” and “regulatory molecule” can be used interchangeably and refer to a molecule that can directly or indirectly affect the production and/or functionality of an effector molecule or effector cell. Signal molecules can be enzymes or other types of biomolecules that can act as a direct ligand on a target cell or they may influence the levels or functionality of a downstream ligand or a receptor for a ligand.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types, ex vivo preparations, and/or a sample of biological materials such as tissue and/or biological fluids.

As used herein, the term “target cell” refers to one or more cells and/or cell types that are deleteriously affected, either directly or indirectly, by a dysregulated immune system and/or a disease process. The term “target cell” also refers to cells that are not deleteriously affected but that are cells in which it is desired that the agent interacts.

As used herein, the term “therapeutically effective amount” refers to the amount of the agent used that is of sufficient quantity to ameliorate, treat and/or inhibit one or more of a disease, disorder or a symptom thereof The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “treat”, “treatment” and “treating” refer to obtaining a desired pharmacologic and/or physiologic, effect. The effect may be prophylactic in terms of completely or partially preventing an occurrence of a disease, disorder or symptom thereof and/or the effect may be therapeutic in providing a partial or complete amelioration or inhibition of a disease, disorder, or symptom thereof. Additionally, the term “treatment” refers to any treatment of a disease, disorder, or symptom thereof. In a subject and includes: (a) preventing the disease from occurring in a subject which may be predisposed to the disease but has not yet been diagnosed as having it; (b) inhibiting the disease, i.e., arresting its development; and, (c) ameliorating the disease.

As used herein., the teens “unit dosage form” and “unit dose” refer to a physically discrete unit that is suitable as a unitary dose for patients. Each unit contains a predetermined quantity of the agent and optionally, one or more able pharmaceutically acceptable carriers, one or more excipients, one or more additional active ingredients, or combinations thereof. The amount of agent within each unit is a therapeutically effective amount.

In embodiments of the present disclosure, the pharmaceutical compositions disclosed herein comprise an agent as described above in a total amount by weight of the composition of about 0.1%, to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 1.6%, about 3.7%, about 3.8%, about 3.9%, about 4%, about 4.1%, about 4.2%, about 4.3% about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.1%, about 8.9%, about 9%, about 9.1%, about 9.2%, about 9.3%, about 9.4%, about 9.5%, about 9.6%, about 9.7%, about 9.8%, about 9.9%, about 10%, about 1.1%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be Included in the smaller ranges, and are also, encompassed within the disclosure, subject to any specifically excluded limit in the stated range. Where the stated range includes one or both of the limits, ranges excluding either or both of those included limits are also included in the disclosure.

In some embodiments of the present disclosure, an agent is a plasmid vector for introducing into a target cell for reproduction or transcription of an insert that comprises one or more nucleotide sequences that are carried within the plasmid vector. In some embodiments of the present disclosure, the plasmid sector is a viral vector. In some embodiments of the present disclosure, the vector is an adeno-associated virus vector.

In some embodiments of the present disclosure, the insert comprises one or more nucleotide sequences that encode for production of a protein. The protein is substantially similar, or substantially the same, as a translational product of a gene whose expression results in increased production of a Bevacizumab-like protein (BLP) that can reduce the formation of new blood vessels in high metabolic rate cells, such as cancer tumor cells. In some embodiments GC the present disclosure, the BLP may be susceptible to one or more post-translational modification processes to create a BLP product that can bind with and inhibit VEGF. For clarity, references to BLP herein include references to. a BLP product.

In some embodiments of the present disclosure, the BLP is substantially similar, or substantially the same, functionality as Bevacizumab that a subject could receive from an exogenous source.

The present disclosure relates to one or more agents, therapies, treatments, and methods of use of the agents and/or therapies and/or treatments for initiating or upregulating production of the BLP. Some embodiments of the present disclosure relate to methods for making a complex between at least one particle of an agent and at least one target cell of a subject for initiating or increasing production of the BLP. Therefore, the administration of the one or more vectors may increase the production of the precursor protein within one or more of a subject's cells. As such, the embodiments of the present disclosure can be used as a therapy or a treatment for a subject that has a condition whereby the high metabolic rate cells, such as tumor cells, are present.

In some embodiments of the present disclosure, the agent can be administered to the subject by an intravenous route, an intramuscular route, an intraperitoneal route, an intrathecal route, an intravesical route, a topical route, an intranasal route, a transmucosal route, a pulmonary route, and combinations thereto.

In some embodiments f the present disclosure, the agent can be administered the subject by pipetting a dose of the agent into an in vitro cell culture, perfusing, or immersing an ex vivo cell or tissue preparation with a solution that comprises the agent, mixing a biological fluid sample with a solution or substrate that comprises the agent, or combinations thereof.

Some embodiments of the present disclosure relate to an agent that can be administered to a subject with a condition that could benefit from an endogenous source of BLP. When a therapeutically effective amount of the agent is administered to the subject, one or more of the subject's cells may increase the translational production of the BLP.

In some embodiments of the present disclosure, the agent is a vector used for gene therapy. The gene therapy is useful for increasing the subject's endogenous production of the BLP. For example, the vector can contain one or more nucleotide sequences that that cause increased production of the BLP in the subject's cells where the vector is expressed.

In some embodiments of the present disclosure, the vector used for gene therapy is a virus that can be enveloped or not, replication effective or not, or combinations thereof. In some embodiments of the present disclosure, the vector is a virus that is not enveloped and not replication effective. In some embodiments of the present disclosure, the vector is a virus of the Paroviridae family. In some embodiments of the present disclosure, the vector is a virus of the genus Dependoparvaovirus. In some embodiments of the present disclosure, the vector is an adeno-associated virus (AAV). In some embodiments of the present disclosure, the vector is a recombinant AAV.

The embodiments of the present disclosure also relate to administering a therapeutically effective amount of the agent. In some embodiments of the present disclosure, the therapeutically effective amount of the agent that is administered to a patient is between about 10 and about 1×10¹⁶ TCID₅₀/kg (50% tissue culture infective dose per kilogram of the patient's body weight). In some embodiments of the present disclosure, the therapeutically effective amount of the agent that is administered to the patient is about 1×10¹³ TCID₅₀/kg. In some embodiments of the present disclosure, the therapeutically effective amount of the agent that is administered to a patient is measured in TPC/kg (total particle count of the agent per kilogram of the patient's body weight). lit some embodiments the therapeutically effective amount of the agent is between about 10 and about 1×10¹⁶ TCP/kg.

Some embodiments of the present disclosure relate to a method for making a complex within a subject. The method comprises a step of administering a therapeutically effective amount of the agent to the subject. The complex comprises at least one particle of the agent and one or more target cells. When the complex is formed, it affects a change in the metabolism of the one or more target cells, which results in the target cells starting and/or upregulating the production of the BLP. Examples of a target cell include both capable cells and incapable cells, such as but not limited to: an innate immune cell; an acquired immune cell; an adrenal gland cell; a bile duct cell; a chondrocyte; a cochlear cell; a corneal cell; an endocardium cell; an endometrial cell; an endothelial cell; an epithelial cell; a fibroblast; a hair follicle cell; a hepatocyte; a lymph node cell; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a sacral cell; a Schwann cell; a T cell; a testicular tissue cell; a thyroid gland cell; a uveal cell; a tumor cell; and/or combinations thereof.

Some embodiments of the present disclosure relate to a therapy, or method of treating a condition, that can be administered to a subject with the condition. The therapy comprises a step of administering to the subject a therapeutically effective amount of an agent that will upregulate the subject's production of the BLP. The increased production of the BLP may result in increased levels of functional and bioavailable BLP, which may reduce deleterious effects of the condition upon the subject. For example, the BLP may reduce the development of new blood vessels in high metabolic-rate cells, such as tumor cells.

Below are examples of nucleotide sequences which may be present in the insert. As will be appreciated by those skilled in the art, minor modifications, substitutions or replacements of a select few nucleotides or amino acids in the sequences provided below will not substantially impact the physiologic or biologic effect of such modified sequences, as compared to the sequences provided herein below. Any such modified sequences are also contemplated by the present disclosure.

Some embodiments of the present disclosure relate to the following nucleotide sequence SEQ ID No. 1 (an inverted terminal repeat):

| | |
|---|---|
| ctgcgcgctc gctcgctcac tgaggccgcc cgggcaaagc ccgggcgtcg ggcgaccttt | 60 |
| ggtcgcccgg cctcagtgag cgagcgagcg cgcagagagg gagtggccaa ctccatcact | 120 |
| aggggttcct | 130 |

Some embodiments of the present disclosure relate to the following nucleotide sequence SEQ ID No. 2 (an inverted terminal repeat):

| | |
|---|---|
| aggaacccct agtgatggag ttggccactc cctctctgcg cgctcgctcg ctcactgagg | 60 |
| ccgggcgacc aaaggtcgcc cgacgcccgg gctttgcccg ggcggcctca gtgagcgagc | 120 |
| gagcgcgc | 128 |

Some embodiments of the present disclosure relate to the following nucleotide sequence SEQ ID No. 3 (a CAST promoter):

| | |
|---|---|
| ggagttccgc gttacataac ttacggtaaa tggcccgcct ggctgaccgc ccaacgaccc | 60 |
| ccgcccattg acgtcaataa tgacgtatgt tcccatagta acgccaatag ggactttcca | 120 |
| ttgacgtcaa tgggtggagt atttacggta aactgcccac ttggcagtac atcaagtgta | 180 |
| tcatatgcca agtacgcccc ctattgacgt caatgacggt aaatggcccg cctggcatta | 240 |
| tgcccagtac atgaccttat gggactttcc tacttggcag tacatctacg tattagtcat | 300 |
| cgctattacc atggtcgagg tgagccccac gttctgcttc actctcccca tctccccccc | 360 |
| ctccccaccc ccaattttgt atttatttat tttttaatta ttttgtgcag cgatgggggc | 420 |
| gggggggggg gggggcgcgc gccaggcggg gcggggcggg gcgaggggcg gggcggggcg | 480 |
| aggcggagag gtgcggcggc agccaatcag agcggcgcgc tccgaaagtt tccttttatg | 540 |
| gcgaggcggc ggcggcggcg gccctataaa aagcgaagcg cgcggcgggc gggagtcgct | 600 |
| gcgcgctgcc ttcgccccgt gccccgctcc gccgccgcct cgcgccgccc gccccggctc | 660 |
| tgactgaccg cgttactaaa acaggtaagt ccggcctccg cgccgggttt tggcgcctcc | 720 |
| cgcgggcgcc cccctcctca cggcgagcgc tgccacgtca gacgaagggc gcagcgagcg | 780 |
| tcctgatcct tccgcccgga cgctcaggac agcggcccgc tgctcataag actcggcctt | 840 |
| agaaccccag tatcagcaga aggacatttt aggacgggac ttgggtgact ctagggcact | 900 |
| ggttttcttt ccagagagcg gaacaggcga ggaaaagtag tcccttctcg gcgattctgc | 960 |
| ggagggatct ccgtggggcg gtgaacgccg atgatgcctc tactaaccat gttcatgttt | 1020 |
| tctttttttt tctacaggtc ctgggtgacg aacag | 1055 |

Some embodiments of the present disclosure relate to the following nucleotide sequence SEQ ID No. 4 (a variable heavy-chain portion);

| | |
|---|---|
| acagctcgtt gaaagcggcg gtggactggt gcagccaggg gggtctttgc gactgtcttg | 60 |
| tgccgcatcc ggttatactt ttactaatta tggaatgaac tgggtacggc aggcccctgg | 120 |
| gaagggtctg gaatgggtag gttggatcaa tacctataca ggtgaaccta cctatgctgc | 180 |
| cgacttcaaa aggcggttca cattcagtct ggatactagc aaaagcaccg catacctcca | 240 |
| gatgaactcc ctgcgcgcag aggacactgc tgtgtactat tgtgccaagt acccacacta | 300 |
| ctacggttca tcccactggt atttcgatgt ttggggtcag ggaaccctcg ttacagttag | 360 |
| tagtgcg | 367 |

Some embodiments of the present disclosure relate to the following nucleotide sequence SEQ ID No. 5 (a variable light-chain portion):

| | |
|---|---|
| gatatacaga tgactcaatc tccttctagc ctgtccgcca gcgtggggga ccgagtgaca | 60 |
| atcacttgca gcgccagtca agatatttcc aattacttga attggtacca acagaagcct | 120 |
| ggaaaagcac ccaaggtgtt gatctacttt acctcttctc ttcattctgg tgtgccaagc | 180 |
| agattttctg gctctggtag tgggactgat ttcactctta ctatcagcag cttgcaacct | 240 |
| gaggatttcg caacctacta ttgtcaacag tattctactg tgccttggac atttggtcag | 300 |
| ggaactaagg tagaaatcaa acgc | 324 |

Some embodiments of the present disclosure relate to the following nucleotide sequence SEQ ID No. 6 (a human IgG-1 constant heavy-chain portion):

| | |
|---|---|
| agcaccaagg gcccatcggt cttccccctg gcaccctcct ccaagagcac ctctgggggc | 60 |
| acagcggccc tgggctgcct ggtcaaggac tacttccccg aaccggtgac ggtgtcgtgg | 120 |
| aactcaggcg ccctgaccag cggcgtgcac accttcccgg ctgtcctaca gtcctcagga | 180 |
| ctctactccc tcagcagcgt ggtgaccgtg ccctccagca gcttgggcac ccagacctac | 240 |
| atctgcaacg tgaatcacaa gcccagcaac accaaggtgg acaagaaagt tgagcccaaa | 300 |
| tcttgtgaca aaactcacac atgcccaccg tgcccagcac ctgaactcct ggggggaccg | 360 |
| tcagtcttcc tcttcccccc aaaacccaag gacaccctca tgatctcccg gacccctgag | 420 |
| gtcacatgcg tggtggtgga cgtgagccac gaagaccctg aggtcaagtt caactggtac | 480 |
| gtggacggcg tggaggtgca taatgccaag acaaagccgc gggaggagca gtacaacagc | 540 |
| acgtaccgtg tggtcagcgt cctcaccgtc ctgcaccagg actggctgaa tggcaaggag | 600 |
| tacaagtgca aggtctccaa caaagccctc ccagccccca tcgagaaaac catctccaaa | 660 |
| gccaaagggc agccccgaga accacaggtg tacaccctgc ccccatcccg ggaggagatg | 720 |
| accaagaacc aggtcagcct gacctggctg gtcaaaggct tctatcccag cgacatcgcc | 780 |
| gtggagtggg agagcaatgg gcagccggag aacaactaca agaccacgcc tcccgtgctg | 840 |
| gactccgacg gctccttctt cctctacagc aagctcaccg tggacaagag caggtggcag | 900 |
| caggggaacg tcttctcatg ctccgtgatg catgaggctc tgcacaacca ctacacgcag | 960 |
| aagagcctct ccctgtctcc gggtaaa | 987 |

Some embodiments of the present disclosure relate to the following nucleotide sequence SEQ ID No. 7 (a human IgG-1 Kappa light-chain portion):

| | |
|---|---|
| ggtcagccca aggctgcccc ctcggtcact ctgttcccgc cctcctctga ggagcttcaa | 60 |
| gccaacaagg ccacactggt gtgtctcata agtgacttct acccgggagc cgtgacagtg | 120 |
| gcctggaagg cagatagcag ccccgtcaag gcgggagtgg agaccaccac accctccaaa | 180 |
| caaagcaaca acaagtacgc ggccagcagc tatctgagcc tgacgcctga gcagtggaag | 240 |
| tcccacagaa gctacagctg ccaggtcacg catgaaggga gcaccgtgga gaagacagtg | 300 |
| gcccctacag aatgttcata g | 321 |

Some embodiments of the present disclosure relate to the following nucleotide sequence SEQ ID No. 8 (a Woodchuck Hepatitis Posttranslational Regulatory Element (WPRE) portion):

| | |
|---|---|
| aatcaacctc tggattacaa aatttgtgaa agattgactg gtattcttaa ctatgttgct | 60 |
| ccttttacgc tatgtggata cgctgcttta atgcctttgt atcatgctat tgcttcccgt | 120 |
| atggctttca ttttctcctc cttgtataaa tcctggttgc tgtctcttta tgaggagttg | 180 |
| tggcccgttg tcaggcaacg tggcgtggtg tgcactgtgt ttgctgacgc aacccccact | 240 |
| ggttggggca ttgccaccac ctgtcagctc ctttccggga ctttcgcttt ccccctccct | 300 |
| attgccacgg cggaactcat cgccgcctgc cttgcccgct gctggacagg ggctcggctg | 360 |
| ttgggcactg acaattccgt ggtgttgtcg gggaaatcat cgtcctttcc ttggctgctc | 420 |
| gcctgtgttg ccacctggat tctgcgcggg acgtccttct gctacgtccc ttcggccctc | 480 |
| aatccagcgg accttccttc ccgcggcctg ctgccggctc tgcggcctct tccgcgtctt | 540 |
| cgccttcgcc ctcagacgag tcggatctcc ctttgggccg cctccccgc | 589 |

Some embodiments of the present disclosure relate to the following nucleotide sequence SEQ ID No. 9 (an AAV vector):

| | |
|---|---|
| ctgcgcgctc gctcgctcac tgaggccgcc cgggcaaagc ccgggcgtcg ggcgaccttt | 60 |
| ggtcgcccgg cctcagtgag cgagcgagcg cgcagagagg gagtggccaa ctccatcact | 120 |
| aggggttcct tgtagttaat gattaacccg ccatgctact tatctacgta gccatgctct | 180 |
| aggacattga ttattgacta gtggagttcc gcgttacata acttacggta aatggcccgc | 240 |
| ctggctgacc gcccaacgac ccccgcccat tgacgtcaat aatgacgtat gttcccatag | 300 |
| taacgccaat agggactttc cattgacgtc aatgggtgga gtatttacgg taaactgccc | 360 |
| acttggcagt acatcaagtg tatcatatgc caagtacgcc ccctattgac gtcaatgacg | 420 |
| gtaaatggcc cgcctggcat tatgcccagt acatgacctt atgggacttt cctacttggc | 480 |
| agtacatcta cgtattagtc atcgctatta ccatggtcga ggtgagcccc acgttctgct | 540 |
| tcactctccc catctccccc ccctccccac ccccaatttt gtatttattt attttttaat | 600 |
| tattttgtgc agcgatgggg gcgggggggg gggggggcgc gcgccaggcg gggcggggcg | 660 |
| gggcgagggg cggggcgggg cgaggcggag aggtgcggcg gcagccaatc agagcggcgc | 720 |
| gctccgaaag tttcctttta tggcgaggcg gcggcggcgg cggccctata aaaagcgaag | 780 |
| cgcgcggcgg gcgggagtcg ctgcgcgctg ccttcgcccc gtgccccgct ccgccgccgc | 840 |
| ctcgcgccgc ccgccccggc tctgactgac cgcgttacta aaacaggtaa gtccggcctc | 900 |
| cgcgccgggt tttggcgcct cccgcgggcg cccccctcct cacggcgagc gctgccacgt | 960 |
| cagacgaagg gcgcagcgag cgtcctgatc cttccgcccg gacgctcagg acagcggccc | 1020 |
| gctgctcata agactcggcc ttagaacccc agtatcagca gaaggacatt ttaggacggg | 1080 |
| acttgggtga ctctagggca ctggttttct ttccagagag cggaacaggc gaggaaaagt | 1140 |
| agtcccttct cggcgattct gcggagggat ctccgtgggg cggtgaacgc cgatgatgcc | 1200 |
| tctactaacc atgttcatgt tttctttttt tttctacagg tcctgggtga cgaacagggt | 1260 |
| accataactt cgtatattgt atgctatacg aagttatgcc accatggcga cgggttcaag | 1320 |
| aacttcccta cttcttgcat ttggcctgct ttgtttgccg tggttacagg agggctcggc | 1380 |
| agaagtacag ctcgttgaaa gcggcggtgg actggtgcag ccaggggggt ctttgcgact | 1440 |
| gtcttgtgcc gcatccggtt atacttttac taattatgga atgaactggg tacggcaggc | 1500 |
| ccctgggaag ggtctggaat gggtaggttg gatcaatacc tatacaggtg aacctaccta | 1560 |
| tgctgccgac ttcaaaaggc ggttcacatt cagtctggat actagcaaaa gcaccgcata | 1620 |
| cctccagatg aactccctgc gcgcagagga cactgctgtg tactattgtg ccaagtaccc | 1680 |
| acactactac ggttcatccc actggtattt cgatgtttgg ggtcagggaa ccctcgttac | 1740 |
| agttagtagt gcgagcacca agggcccatc ggtcttcccc ctggcaccct cctccaagag | 1800 |
| cacctctggg ggcacagcgg ccctgggctg cctggtcaag gactacttcc ccgaaccggt | 1860 |
| gacggtgtcg tggaactcag gcgccctgac cagcggcgtg cacaccttcc cggctgtcct | 1920 |
| acagtcctca ggactctact ccctcagcag cgtggtgacc gtgccctcca gcagcttggg | 1980 |
| cacccagacc tacatctgca acgtgaatca caagcccagc aacaccaagg tggacaagaa | 2040 |
| agttgagccc aaatcttgtg acaaaactca cacatgccca ccgtgcccag cacctgaact | 2100 |
| cctgggggga ccgtcagtct tcctcttccc cccaaaaccc aaggacaccc tcatgatctc | 2160 |
| ccggacccct gaggtcacat gcgtggtggt ggacgtgagc cacgaagacc ctgaggtcaa | 2220 |
| gttcaactgg tacgtggacg gcgtggaggt gcataatgcc aagacaaagc cgcgggagga | 2280 |
| gcagtacaac agcacgtacc gtgtggtcag cgtcctcacc gtcctgcacc aggactggct | 2340 |
| gaatggcaag gagtacaagt gcaaggtctc caacaaagcc ctcccagccc ccatcgagaa | 2400 |
| aaccatctcc aaagccaaag ggcagccccg agaaccacag gtgtacaccc tgcccccatc | 2460 |
| ccgggaggag atgaccaaga accaggtcag cctgacctgc ctggtcaaag gcttctatcc | 2520 |
| cagcgacatc gccgtggagt gggagagcaa tgggcagccg gagaacaact acaagaccac | 2580 |
| gcctcccgtg ctggactccg acggctcctt cttcctctac agcaagctca ccgtggacaa | 2640 |
| gagcaggtgg cagcagggga acgtcttctc atgctccgtg atgcatgagg ctctgcacaa | 2700 |
| ccactacacg cagaagagcc tctccctgtc tccgggtaaa cgaaaaagaa gatcaggttc | 2760 |
| gggtgcgcca gtaaagcaga cattaaactt tgatttgctg aaacttgcag gtgatgtaga | 2820 |
| gtcaaatcca ggtccaatgg caacagggag ccgaacctct ctgctccttg ctttcgggct | 2880 |
| cctttgccta ccgtggctcc aagagggctc ggcagatata cagatgactc aatctccttc | 2940 |
| tagcctgtcc gccagcgtgg gggaccgagt gacaatcact tgcagcgcca gtcaagatat | 3000 |
| ttccaattac ttgaattggt accaacagaa gcctggaaaa gcacccaagg tgttgatcta | 3060 |
| ctttacctct tctcttcatt ctggtgtgcc aagcagattt tctggctctg gtagtgggac | 3120 |
| tgatttcact cttactatca gcagcttgca acctgaggat ttcgcaacct actattgtca | 3180 |
| acagtattct actgtgcctt ggacatttgg tcagggaact aaggtagaaa tcaaacgcgg | 3240 |
| tcagcccaag gctgccccct cggtcactct gttcccgccc tcctctgagg agcttcaagc | 3300 |
| caacaaggcc acactggtgt gtctcataag tgacttctac ccgggagccg tgacagtggc | 3360 |
| ctggaaggca gatagcagcc ccgtcaaggc gggagtggag accaccacac cctccaaaca | 3420 |
| aagcaacaac aagtacgcgg ccagcagcta tctgagcctg acgcctgagc agtggaagtc | 3480 |
| ccacagaagc tacagctgcc aggtcacgca tgaagggagc accgtggaga agacagtggc | 3540 |
| ccctacagaa tgttcataga taacttcgta taatgtatgc tatacgaagt tattctagaa | 3600 |
| taatcaacct ctggattaca aaatttgtga aagattgact ggtattctta actatgttgc | 3660 |
| tccttttacg ctatgtggat acgctgcttt aatgcctttg tatcatgcta ttgcttcccg | 3720 |
| tatggctttc attttctcct ccttgtataa atcctggttg ctgtctcttt atgaggagtt | 3780 |
| gtggcccgtt gtcaggcaac gtggcgtggt gtgcactgtg tttgctgacg caacccccac | 3840 |
| tggttggggc attgccacca cctgtcagct cctttccggg actttcgctt tccccctccc | 3900 |
| tattgccacg gcggaactca tcgccgcctg ccttgcccgc tgctggacag gggctcggct | 3960 |
| gttgggcact gacaattccg tggtgttgtc ggggaaatca tcgtcctttc cttggctgct | 4020 |
| cgcctgtgtt gccacctgga ttctgcgcgg gacgtccttc tgctacgtcc cttcggccct | 4080 |
| caatccagcg gaccttcctt cccgcggcct gctgccggct ctgcggcctc ttccgcgtct | 4140 |
| tcgccttcgc cctcagacga gtcggatctc cctttgggcc gcctccccgc ctaagcttat | 4200 |
| cgataccgtc gagatctaac ttgtttattg cagcttataa tggttacaaa taaagcaata | 4260 |
| gcatcacaaa tttcacaaat aaagcatttt tttcactgca ttctagttgt ggtttgtcca | 4320 |
| aactcatcaa tgtatcttat catgtctgga tctcgacctc gactagagca tggctacgta | 4380 |
| gataagtagc atggcgggtt aatcattaac tacaaggaac ccctagtgat ggagttggcc | 4440 |
| actccctctc tgcgcgctcg ctcgctcact gaggccgggc gaccaaaggt cgcccgacgc | 4500 |
| ccgggctttg cccgggcggc ctcagtgagc gagcgagcgc gccagctggc gtaatagcga | 4560 |
| agaggcccgc accgatcgcc cttcccaaca gttgcgcagc ctgaatggcg aatggaattc | 4620 |
| cagacgattg agcgtcaaaa tgtaggtatt tccatgagcg tttttcctgt tgcaatggct | 4680 |
| ggcggtaata ttgttctgga tattaccagc aaggccgata gtttgagttc ttctactcag | 4740 |
| gcaagtgatg ttattactaa tcaaagaagt attgcgacaa cggttaattt gcgtgatgga | 4800 |
| cagactcttt tactcggtgg cctcactgat tataaaaaca cttctcagga ttctggcgta | 4860 |
| ccgttcctgt ctaaaatccc tttaatcggc ctcctgttta gctcccgctc tgattctaac | 4920 |
| gaggaaagca cgttatacgt gctcgtcaaa gcaaccatag tacgcgccct gtagcggcgc | 4980 |
| attaagcgcg gcgggtgtgg tggttacgcg cagcgtgacc gctacacttg ccagcgccct | 5040 |
| agcgcccgct cctttcgctt tcttcccttc ctttctcgcc acgttcgccg gctttccccg | 5100 |
| tcaagctcta aatcgggggc tccctttagg gttccgattt agtgctttac ggcacctcga | 5160 |
| ccccaaaaaa cttgattagg gtgatggttc acgtagtggg ccatcgccct gatagacggt | 5220 |
| ttttcgccct ttgacgttgg agtccacgtt ctttaatagt ggactcttgt tccaaactgg | 5280 |
| aacaacactc aaccctatct cggtctattc ttttgattta taagggattt tgccgatttc | 5340 |
| ggcctattgg ttaaaaaatg agctgattta acaaaaattt aacgcgaatt ttaacaaaat | 5400 |
| attaacgttt acaatttaaa tatttgctta tacaatcttc ctgtttttgg ggcttttctg | 5460 |
| attatcaacc ggggtacata tgattgacat gctagtttta cgattaccgt tcatcgattc | 5520 |
| tcttgtttgc tccagactct caggcaatga cctgatagcc tttgtagaga cctctcaaaa | 5580 |
| atagctaccc tctccggcat gaatttatca gctagaacgg ttgaatatca tattgatggt | 5640 |
| gatttgactg tctccggcct ttctcacccg tttgaatctt tacctacaca ttactcaggc | 5700 |
| attgcattta aaatatatga gggttctaaa aatttttatc cttgcgttga aataaaggct | 5760 |
| tctcccgcaa aagtattaca gggtcataat gtttttggta caaccgattt agctttatgc | 5820 |
| tctgaggctt tattgcttaa ttttgctaat tctttgcctt gcctgtatga tttattggat | 5880 |
| gttggaattc ctgatgcggt attttctcct tacgcatctg tgcggtattt cacaccgcat | 5940 |
| atggtgcact ctcagtacaa tctgctctga tgccgcatag ttaagccagc cccgacaccc | 6000 |
| gccaacaccc gctgacgcgc cctgacgggc ttgtctgctc ccggcatccg cttacagaca | 6060 |
| agctgtgacc gtctccggga gctgcatgtg tcagaggttt tcaccgtcat caccgaaacg | 6120 |
| cgcgagacga aagggcctcg tgatacgcct atttttatag gttaatgtca tgataataat | 6180 |
| ggtttcttag acgtcaggtg gcacttttcg gggaaatgtg cgcggaaccc ctatttgttt | 6240 |
| atttttctaa atacattcaa atatgtatcc gctcatgaga caataaccct gataaatgct | 6300 |
| tcaataatat tgaaaaagga agagtatgag tattcaacat ttccgtgtcg cccttattcc | 6360 |
| cttttttgcg gcattttgcc ttcctgtttt tgctcaccca gaaacgctgg tgaaagtaaa | 6420 |
| agatgctgaa gatcagttgg gtgcacgagt gggttacatc gaactggatc tcaacagcgg | 6480 |
| taagatcctt gagagttttc gccccgaaga acgttttcca atgatgagca cttttaaagt | 6540 |
| tctgctatgt ggcgcggtat tatcccgtat tgacgccggg caagagcaac tcggtcgccg | 6600 |
| catacactat tctcagaatg acttggttga gtactcacca gtcacagaaa agcatcttac | 6660 |
| ggatggcatg acagtaagag aattatgcag tgctgccata accatgagtg ataacactgc | 6720 |
| ggccaactta cttctgacaa cgatcggagg accgaaggag ctaaccgctt ttttgcacaa | 6780 |
| catgggggat catgtaactc gccttgatcg ttgggaaccg gagctgaatg aagccatacc | 6840 |
| aaacgacgag cgtgacacca cgatgcctgt agcaatggca acaacgttgc gcaaactatt | 6900 |
| aactggcgaa ctacttactc tagcttcccg gcaacaatta atagactgga tggaggcgga | 6960 |
| taaagttgca ggaccacttc tgcgctcggc ccttccggct ggctggttta ttgctgataa | 7020 |
| atctggagcc ggtgagcgtg ggtctcgcgg tatcattgca gcactggggc cagatggtaa | 7080 |
| gccctcccgt atcgtagtta tctacacgac ggggagtcag gcaactatgg atgaacgaaa | 7140 |
| tagacagatc gctgagatag gtgcctcact gattaagcat tggtaactgt cagaccaagt | 7200 |
| ttactcatat atactttaga ttgatttaaa acttcatttt taatttaaaa ggatctaggt | 7260 |
| gaagatcctt tttgataatc tcatgaccaa aatcccttaa cgtgagtttt cgttccactg | 7320 |
| agcgtcagac cccgtagaaa agatcaaagg atcttcttga gatccttttt ttctgcgcgt | 7380 |
| aatctgctgc ttgcaaacaa aaaaaccacc gctaccagcg gtggtttgtt tgccggatca | 7440 |
| agagctacca actctttttc cgaaggtaac tggcttcagc agagcgcaga taccaaatac | 7500 |
| tgtccttcta gtgtagccgt agttaggcca ccacttcaag aactctgtag caccgcctac | 7560 |
| atacctcgct ctgctaatcc tgttaccagt ggctgctgcc agtggcgata agtcgtgtct | 7620 |
| taccgggttg gactcaagac gatagttacc ggataaggcg cagcggtcgg gctgaacggg | 7680 |
| gggttcgtgc acacagccca gcttggagcg aacgacctac accgaactga gatacctaca | 7740 |
| gcgtgagcta tgagaaagcg ccacgcttcc cgaagggaga aaggcggaca ggtatccggt | 7800 |
| aagcggcagg gtcggaacag gagagcgcac gagggagctt ccagggggaa acgcctggta | 7860 |
| tctttatagt cctgtcgggt ttcgccacct ctgacttgag cgtcgatttt tgtgatgctc | 7920 |
| gtcagggggg cggagcctat ggaaaaacgc cagcaacgcg gcctttttac ggttcctggc | 7980 |
| cttttgctgg ccttttgctc gagctgatac tcctgcgtta tcccctgatt ctgtggataa | 8040 |
| ccgtattacc gcctttgagt gagctgatac cgctcgccgc agccgaacga ccgagcgcag | 8100 |
| cgagtcagtg agcgaggaag cggaagagcg cccaatacgc aaaccgcctc tccccgcgcg | 8160 |
| ttggccgatt cattaatgca gcag | 8184 |

Some embodiments of the present disclosure relate to the insert that comprises the nucleotide sequence SEQ ID No. 1, SEQ ID No. 2, SEQ ID No. 3, SEQ ID No. 4, SEQ ID No. 5, SEQ ID No. 6, SEQ ID No. 7, SEQ ID No. 8 or combinations thereof.

Some embodiments of the present disclosure relate to the insert that comprises the nucleotide sequences of SEQ ID No. 4, SEQ ID No. 5, SEQ ID No. 6 and SEQ ID No. 7.

Some embodiments of the present disclosure relate to a composition of matter and/or the agent that comprises the nucleotide sequence of SEQ ID No. 9.

### Example 1

### Expression Cassette

Expression cassettes for expressing the BLP in a subject cell were synthesized by Genscript. Each cassette contained a signal peptide, the precursor protein that may be followed by a self-cleaving 2A peptide sequence, a signal peptide and the human lambda constant domain. The synthesized BLP expression cassettes were cloned into the pAVA-00200 plasmid backbone containing the CAS1 promoter1, multiple cloning site (MCS), Woodchuck Hepatitis Virus post-transcriptional regulatory element (WPRE), Simian virus 40 (SV40) polyadenylation (polyA) sequence all flanked by the AAV2 inverted terminal repeats (ITR). pAVA-00200 was cut with the restriction enzymes KpnI and XbaI in the MCS and separated on a 1% agarose gel. The band of interest was excised and purified using a gel extraction kit. Each BLP expression cassette was amplified by PCR using Taq polymerase and the PCR products were gel purified and the bands on interest were also excised and purified using a gel extraction kit. These PCR products contained the BLP expression cassettes in addition to 15 base pair 5′ and 3′ overhangs that align with the ends of the linearized pAVA-00200 backbone. Using infusion cloning2, the amplified BLP expression cassettes were integrated with the pAVA-00200 backbone via homologous recombination. The resulting, plasmid vectors contained at least the following: 5′ ITR, a CASI promoter, the BLP expression cassette, WPRE, and a 3′ ITR, per SEQ ID No. 9.

### Example 2

### Experimental Data

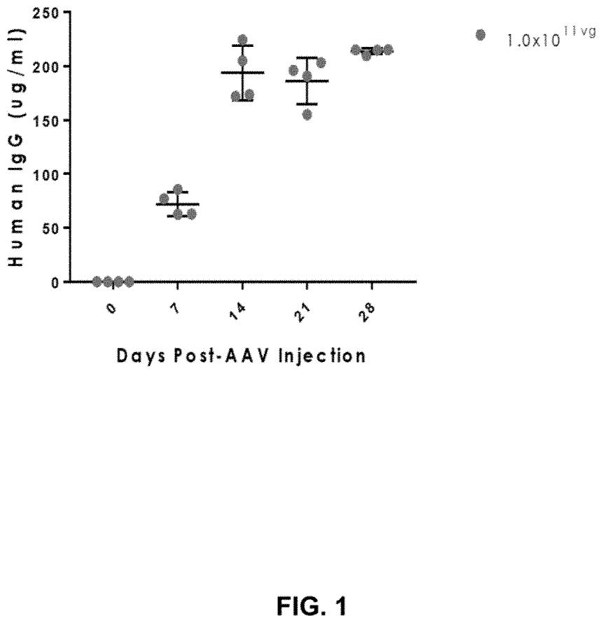

The ovarian bursa of C57BL/6 mice were implanted with 1×10⁶ ID8 epithelial carcinoma cells. About 60 days later, eight mice were administered i.p. 1×10¹¹ 5 mM of phosphate buffered saline (control group) or 1×10 vg of the AAV comprising SEQ ID No. 9 (treatment group). Serum samples were obtained from animals in the control group and the treatment group. The serum samples were analyzed using a quantitative ELISA to measure human IgG-1 levels (as an indicator of BLP production in FIG. 1). The analysis of the serum samples from the animals in the control group showed that no human IgG-1 was detected. FIG. 1 shows the levels of human IgG (μg/mL) detected in the serum samples from the animals in the treatment group up to 28 days following the treatment.

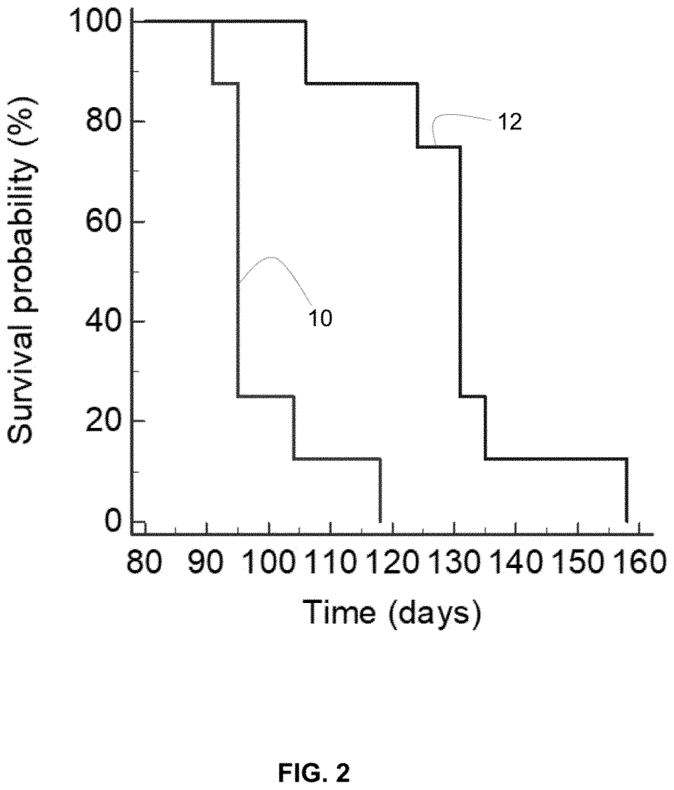

FIG. 2 is a Kaplan-Meier plot showing survival probability for the mice in the control group (shown as line 10) and the mice in the treatment group (shown as line 12). Without being bound by any particular theory, the mice in the treatment group had increased survival over time, as compared to the mice in the control group.

The invention claimed is:

1. A recombinant virus vector (RVV) the RVV comprising:
a. a nucleotide sequence encoding for production of Bevacizumab-like protein (BLP); and
b. an inverted terminal repeat.

2. The RVV of claim 1, wherein the inverted terminal repeat is SEQ ID No. 1 or SEQ ID No. 2.

3. The RVV of claim 1, wherein the inverted to terminal repeat is a first inverted terminal repeat of SEQ ID No. 1 and a second inverted terminal repeat of SEQ ID No. 2, and wherein the nucleotide sequence encoding the BLP is positioned between the first inverted terminal repeat and the second inverted terminal repeat.

4. The RVV of claim 1, wherein the nucleotide sequence encoding for production of the BLP comprises SEQ ID No. 4, SEQ ID No. 5, SEQ ID No. 6 and SEQ ID No. 7.

5. The RVV of claim 1, wherein the RVV is an adeno associated virus vector.

6. A composition that: comprises a nucleotide sequence according to SEQ ID No. 9 that can be expressed in a target cell.

7. A pharmaceutical composition comprising the RVV of claim 1 and one or more pharmaceutically acceptable carriers and/or one or more excipients.

8. A method of making an agent target cell complex, the method comprising a step of administering a recombinant virus vector (RVV) to a target cell for forming the agent tar cell complex, wherein the agent/target cell complex causes the target cell to increase production of a Bevacizumab-like protein (BLP).

9. The method of claim 8, wherein the RVV comprises a nucleotide sequence according to SEQ ID No. 4, SEQ ID No. 5, SEQ ID No. 6 and SEQ ID No. 7 for increasing the target cell's production of the BLP.

10. The method of claim 10, wherein the RVV comprises a nucleotide sequence according to SEQ ID No. 9 for increasing the target cell's production of the BLP.

11. The method of claim 10, wherein the target cell is one or more of an adrenal gland cell; a bile duct cell; a chondrocyte; a cochlear cell; a corneal cell; an endocardium cell; an endometrial cell; an endothelial cell; an epithelial cell; a fibroblast; a hair follicle cell; a hepatocyte; a lymph node cell; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a T cell; a testicular tissue cell; a thyroid gland cell; a uveal cell; a tumor cell; and/or combinations thereof.


SEQUENCE LISTING (9 sequences)

SEQ ID NO: 1 - DNA, 130 bp, Artificial Sequence
  Synthetic Sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 128 bp, Artificial Sequence
  Synthetic Sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 3 - DNA, 1055 bp, Artificial Sequence
  Synthetic Sequence
ggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 4 - DNA, 367 bp, Artificial Sequence
  Synthetic sequence
acagctcgttgaaagcggcggtggactggtgcagccaggggggtctttgcgactgtcttgtgccgcatccggttatacttttactaattatggaatgaactgggtacggcaggcccctgggaagggtctggaatgggtaggttggatcaatacctatacaggtgaacctacctatgctgccgacttcaaaaggcggttcacattcagtctggatactagcaaaagcaccgcatacctccagatgaactccctgcgcgcagaggacactgctgtgtactattgtgccaagtacccacactactacggttcatcccactggtatttcgatgtttggggtcagggaaccctcgttacagttagtagtgcg

SEQ ID NO: 5 - DNA, 324 bp, Artificial Sequence
  Synthetic Sequence
gatatacagatgactcaatctccttctagcctgtccgccagcgtgggggaccgagtgacaatcacttgcagcgccagtcaagatatttccaattacttgaattggtaccaacagaagcctggaaaagcacccaaggtgttgatctactttacctcttctcttcattctggtgtgccaagcagattttctggctctggtagtgggactgatttcactcttactatcagcagcttgcaacctgaggatttcgcaacctactattgtcaacagtattctactgtgccttggacatttggtcagggaactaaggtagaaatcaaacgc

SEQ ID NO: 6 - DNA, 987 bp, Artificial Sequence
  Synthetic Sequence
agcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 7 - DNA, 321 bp, Artificial Sequence
  Synthetic Sequence
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 8 - DNA, 589 bp, Artificial Sequence
  Synthetic Sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 9 - DNA, 8184 bp, Artificial Sequence
  Synthetic Sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggacattgattattgactagtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacagggtaccataacttcgtatattgtatgctatacgaagttatgccaccatggcgacgggttcaagaacttccctacttcttgcatttggcctgctttgtttgccgtggttacaggagggctcggcagaagtacagctcgttgaaagcggcggtggactggtgcagccaggggggtctttgcgactgtcttgtgccgcatccggttatacttttactaattatggaatgaactgggtacggcaggcccctgggaagggtctggaatgggtaggttggatcaatacctatacaggtgaacctacctatgctgccgacttcaaaaggcggttcacattcagtctggatactagcaaaagcaccgcatacctccagatgaactccctgcgcgcagaggacactgctgtgtactattgtgccaagtacccacactactacggttcatcccactggtatttcgatgtttggggtcagggaaccctcgttacagttagtagtgcgagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgaaaaagaagatcaggttcgggtgcgccagtaaagcagacattaaactttgatttgctgaaacttgcaggtgatgtagagtcaaatccaggtccaatggcaacagggagccgaacctctctgctccttgctttcgggctcctttgcctaccgtggctccaagagggctcggcagatatacagatgactcaatctccttctagcctgtccgccagcgtgggggaccgagtgacaatcacttgcagcgccagtcaagatatttccaattacttgaattggtaccaacagaagcctggaaaagcacccaaggtgttgatctactttacctcttctcttcattctggtgtgccaagcagattttctggctctggtagtgggactgatttcactcttactatcagcagcttgcaacctgaggatttcgcaacctactattgtcaacagtattctactgtgccttggacatttggtcagggaactaaggtagaaatcaaacgcggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagataacttcgtataatgtatgctatacgaagttattctagaataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctaagcttatcgataccgtcgagatctaacttgtttattgcagcttataatggttacaaataaagcaatagcatcacaaatttcacaaataaagcatttttttcactgcattctagttgtggtttgtccaaactcatcaatgtatcttatcatgtctggatctcgacctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcag